(19) United States
(12) Patent Application Publication
(10) Pub. No.: US 2011/0190591 A1
(43) Pub. Date: Aug. 4, 2011

(54) SURGICAL RETRACTOR

(76) Inventors: Andrew K. PALMER, Eastham, MA (US); Dale DELLACQUA, Bloomington, IN (US); Jeffrey A. FARRIS, Berne, IN (US)

(21) Appl. No.: 12/696,171

(22) Filed: Jan. 29, 2010

(51) Int. Cl. A61B 1/32

(52) U.S. Cl. 600/226

(57) ABSTRACT

The present invention is a surgical retractor that can be manipulated with only a digit of a single hand. The surgical retractor includes a shaft, which may be cylindrical, with a finger member attached to one side. The finger member may be an arc segment properly sized to keep a digit next to the shaft. Attached to one end of the shaft is a rake head. The rake head consists of three segments attached to one another, the first of which is attached to the shaft. The final segment has a concavity formed upon it to allow another instrument access beneath the surgical retractor. The method of using the surgical retractor is also disclosed.

### CROSS-REFERENCE TO RELATED APPLICATIONS

This application is related to design patent application number 29/354,675 filed on Jan. 27, 2010.

### TECHNICAL FIELD

This invention relates generally to the field of surgical instruments, and more specifically, to the field of surgical retractors.

### BACKGROUND OF THE INVENTION

Surgical retractors are widely used in a variety of operative procedures. These uses may include holding back skin from a wound or incision to allow a surgeon access to the internal aspects of a human body. Other uses of a surgical retractor may include restraining internal organs to allow for surgeon access to a desired location within the body cavity.

Manipulation of a surgical retractor often either requires the surgeon to act alone, or with the assistance of another individual. Having multiple individuals holding a surgical retractor leads to limited visibility and loss of control by the surgeon. As surgical incisions decrease in size, surgical retractors must also decrease in size and be designed to allow for a single operator to use.

Accordingly, it is clear to those skilled in the art that there is a need for improvements to the surgical retractor to allow a surgeon to control and manipulate the instrument while performing surgery with corresponding ancillary instruments. Further, a need exists for a surgical retractor that is constructed to provide the user with the ability to retract all tissue types using a single appendage.

### SUMMARY OF THE INVENTION

The surgical retractor, and the method of using it, of the present invention solve the present problems of multi-user surgical retractors.

The present intention provides, in one aspect, a surgical retractor that includes a shaft with a finger member and a rake head. The finger member being positioned on the superior aspect of the shaft with the rake head being located at the proximal end of the shaft.

The present invention provides in another aspect a surgical method of using the surgical retractor that may include: obtaining a surgical retractor, the surgical retractor comprised of a shaft with a finger member configured to accommodate a guiding digit attached to the shaft, a rake head configured to facilitate entering into an incision connected to the proximal end of the shaft; inserting the rake head into an opening; placing a digit against the finger member; urging the surgical retractor in a primarily axial direction; and engaging the opening such that the width of the opening is increased, thereby allowing the user of the surgical retractor to manipulate additional instruments and granting the user greater control over the surgical retraction.

Other additional features, benefits and advantages of the present invention will become apparent from the following drawings and descriptions of the invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the end of the specification. The foregoing and other objects, features, and advantages of the invention are apparent from the following detailed description taken in conjunction with the accompanying drawings in which:

FIG. 1A is a perspective view of one embodiment of a surgical retractor, in accordance with an aspect of the present invention;

FIG. 1B is a detailed perspective view of the proximal end of the surgical retractor of FIG. 1A, in accordance with an aspect of the present invention;

FIG. 2 is a cross-sectional view of the surgical retractor of FIG. 1A along line 2-2, in accordance with an aspect of the present invention;

FIG. 3A is a cross-sectional view of the proximal end of the surgical retractor of FIG. 1B along line 3A-3A, in accordance with an aspect of the present invention;

FIG. 3B is a cross-sectional view of an alternative embodiment of the proximal end of the surgical retractor of FIG. 1B along line 3B-3B, in accordance with an aspect of the present invention;

FIG. 3C is a cross-sectional view of an alternative embodiment of the proximal end of the surgical retractor of FIG. 1B along line 3C-3C, in accordance with an aspect of the present invention;

FIG. 3D is a cross-sectional view of an alternative embodiment of the proximal end of the surgical retractor of FIG. 1B along line 3D-3D, in accordance with an aspect of the present invention;

FIG. 4A is a side elevational view of the surgical retractor of FIG. 1A, in accordance with an aspect of the invention;

FIG. 4B is a detailed side elevational view of an alternative embodiment of the proximal end of the surgical retractor of FIG. 1A, showing the adjustable rake head, in accordance with an aspect of the invention;

FIG. 4C is an exploded isometric view of an alternative embodiment of the proximal end of the surgical retractor of FIG. 1A, showing the detachable rake head, in accordance with an aspect of the invention;

FIG. 4D is an exploded isometric view of an alternative embodiment of the shaft and finger member of the surgical retractor of FIG. 1A, showing the removably attached finger member, in accordance with an aspect of the invention;

FIG. 4E is a detailed side elevational view of an alternative embodiment of the shaft and finger member of the surgical retractor of FIG. 1A, showing the movably attached finger member, in accordance with an aspect of the invention;

FIG. 5 is a front elevational view of the surgical retractor of FIG. 1A, in accordance with an aspect of the invention;

FIG. 6 is a side elevational view of an alternative embodiment of the surgical retractor of FIG. 1A, showing a straight finger member, in accordance with an aspect of the invention;

FIG. 7 is a side elevational view of an alternative embodiment of the surgical retractor of FIG. 1A, showing an angled finger member, in accordance with an aspect of the invention;

FIG. 8 is a side elevational view of an alternative embodiment of the surgical retractor of FIG. 1A, showing a finger member with multiple members, in accordance with an aspect of the invention;

FIG. 9 is a side elevational view of an alternative embodiment of the surgical retractor of FIG. 1A, showing multiple finger members, in accordance with an aspect of the invention;

FIG. 10 is a perspective view of a user holding the surgical retractor of FIG. 1A, showing a rake head inserted into an opening in the tissue, and a thumb placed against the finger member of the surgical retractor, in accordance with an aspect of the present invention;

FIG. 11 is a perspective view of a user of the surgical retractor of FIG. 1A, showing the surgical retractor urged in a primarily axial direction, and the rake head of the surgical retractor engaging the opening, in accordance with an aspect of the present invention; and

FIG. 12 is a side elevational view of a user axially displacing the surgical retractor of FIG. 1A, showing the rake head engaging the opening, in accordance with an aspect of the present invention.

### DETAILED DESCRIPTION FOR CARRYING OUT THE INVENTION

For the purposes of promoting an understanding of the principles of the surgical retractor and a method of using the surgical retractor, reference will now be made to the embodiments, or examples, illustrated in the drawings and specific language will be used to describe these. It will nevertheless be understood that no limitation of the scope of the invention is thereby intended. Any alterations and further modifications in the described embodiments, and any further applications of the principles of the invention as described herein are contemplated as would normally occur to one skilled in the art to which the surgical retractor invention relates.

In this detailed description and the following claims, the words proximal, distal, anterior, posterior, medial, lateral, superior and inferior are defined by their standard usage for indicating a particular part of a surgical retractor or surgical opening according to the relative disposition of the surgical retractor, surgical opening or directional terms of reference. For example, “proximal” means the portion of the surgical retractor positioned nearest the torso, while “distal” indicates the part of the surgical retractor farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the terms “surgical retractor” and “retractor” may be used interchangeably as they essentially describe the same type of surgical instrument.

Generally stated, disclosed herein is a surgical retractor for use in surgical procedures. The surgical retractor shown herein is intended for example purposes only, as many alterations would occur to one skilled in the art, and are contemplated as a part of the invention. The surgical retractor generally includes a shaft, a finger member, and a head rake. Stated generally, in a method of using the surgical retractor, the head rake may be inserted into an opening, a digit may be placed against the finger member, the surgical retractor may then be urged or pulled in a primarily axial direction, and the opening may be engaged and widened when tensed.

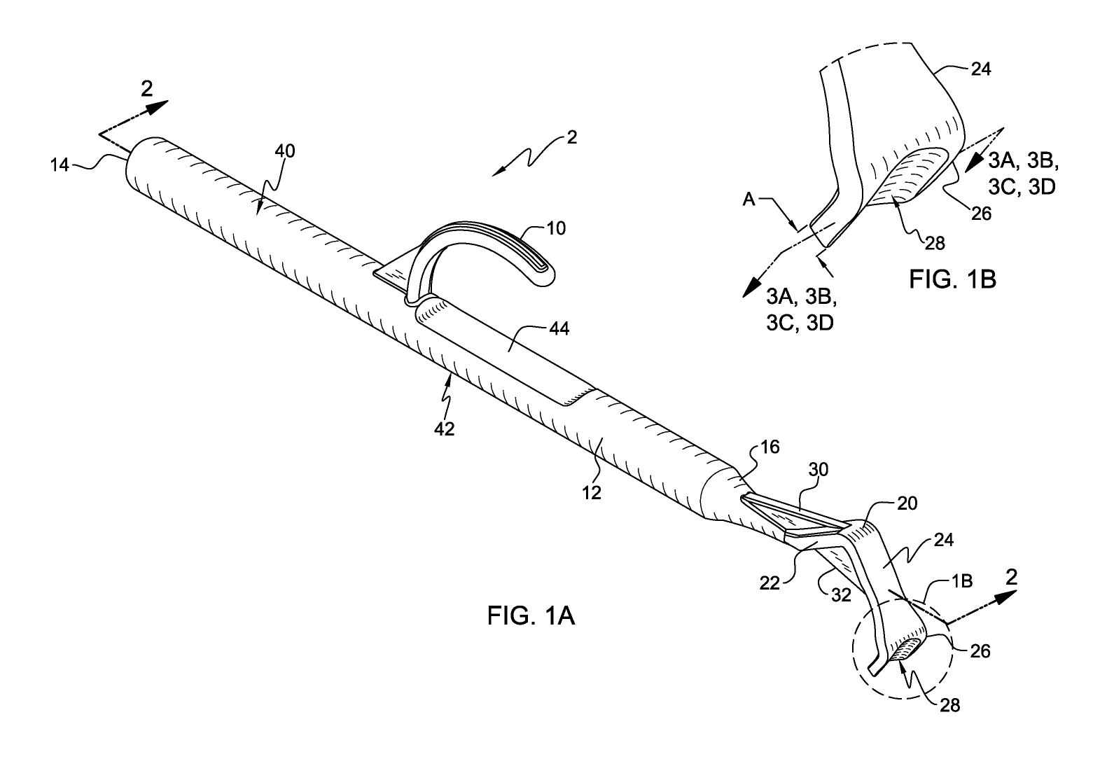

The present invention comprises a surgical retractor, which may be manipulated by a single hand concurrently with another instrument, and is identified in FIGS. 1A-12 by reference numeral 2.

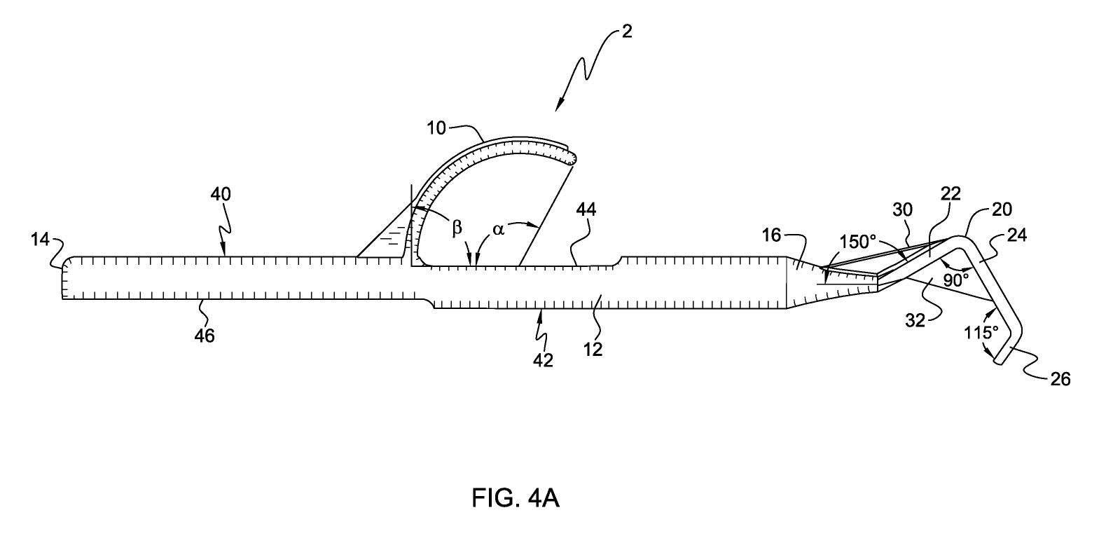

Referring to FIG. 1A, surgical retractor 2 comprises a finger member 10, a shaft 12, and a rake head 20. Shaft 12 has a distal end 14 and a proximal end 16. Rake head 20 is attached to proximal end 16. Shaft 12 has a superior surface 40 and an inferior surface 42. In the embodiment shown in FIGS. 1A and 4A, finger member 10 is attached to superior surface 40. However, in alternative embodiments, finger member 10 may be attached to inferior surface 42. In other embodiments, finger member 10 may be attached to any location between superior surface 40 and inferior surface 42 or along the circumference of shaft 12. Finger member 10 may be attached to any location on shaft 12. In the embodiment shown in FIGS. 1A and 4A, finger member 10 is attached to shaft 12 on superior surface 40 intermediate of distal end 14 and proximal end 16. However, finger member 10 may be attached closer to either distal end 14 or to proximal end 16. It is also contemplated that finger member 10 may be attached to either distal end 14 or proximal end 16.

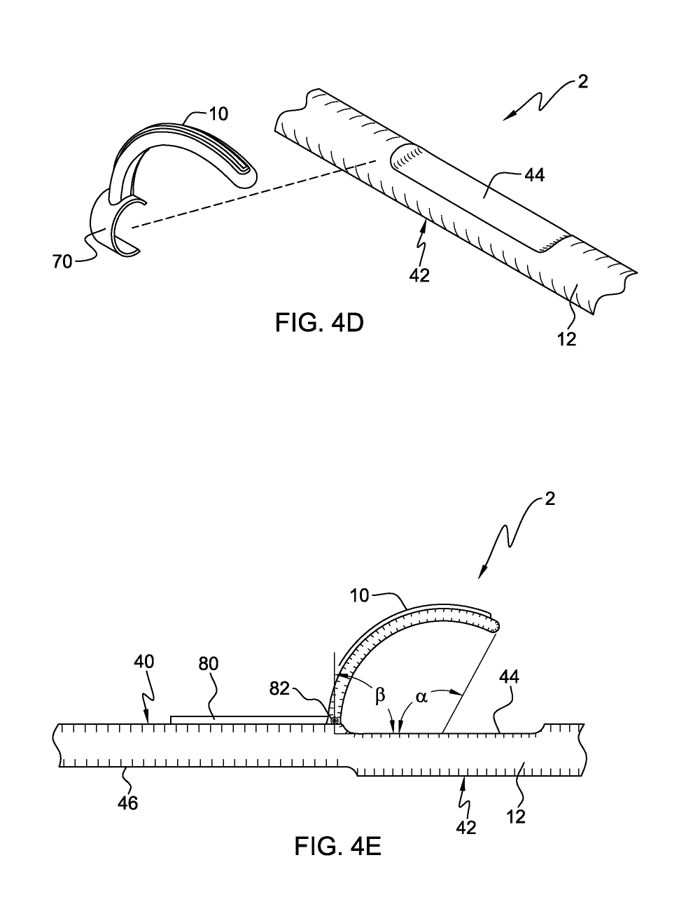

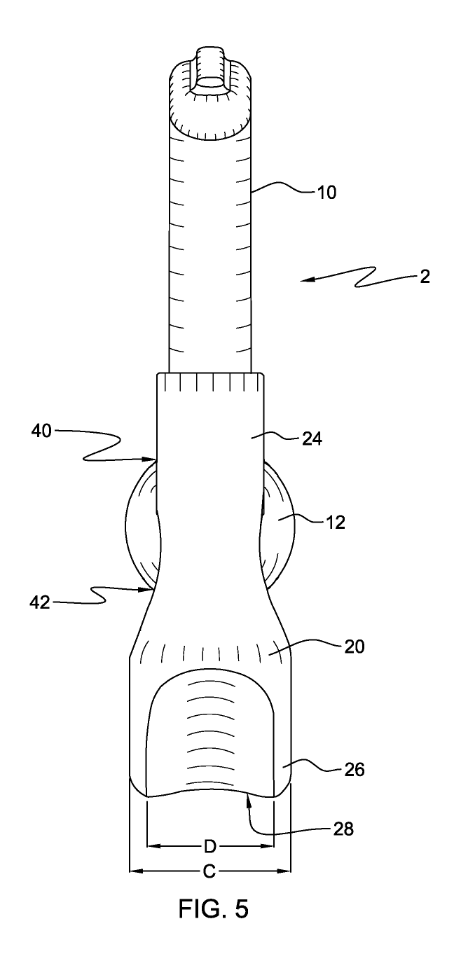

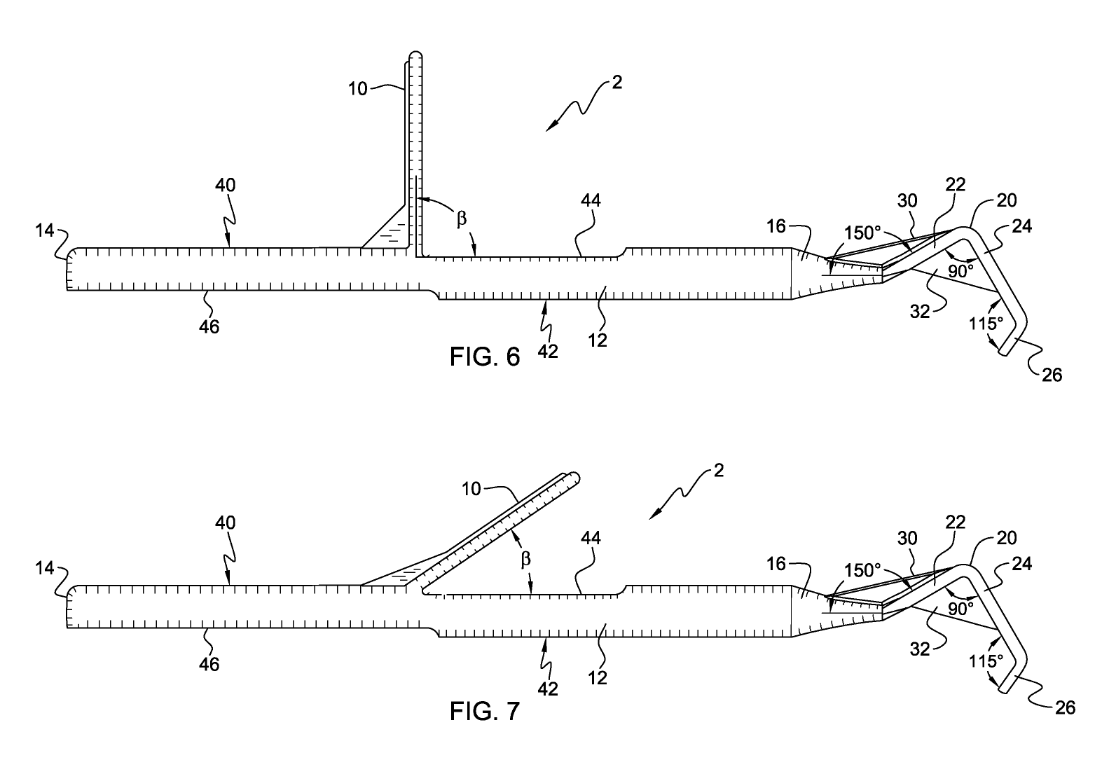

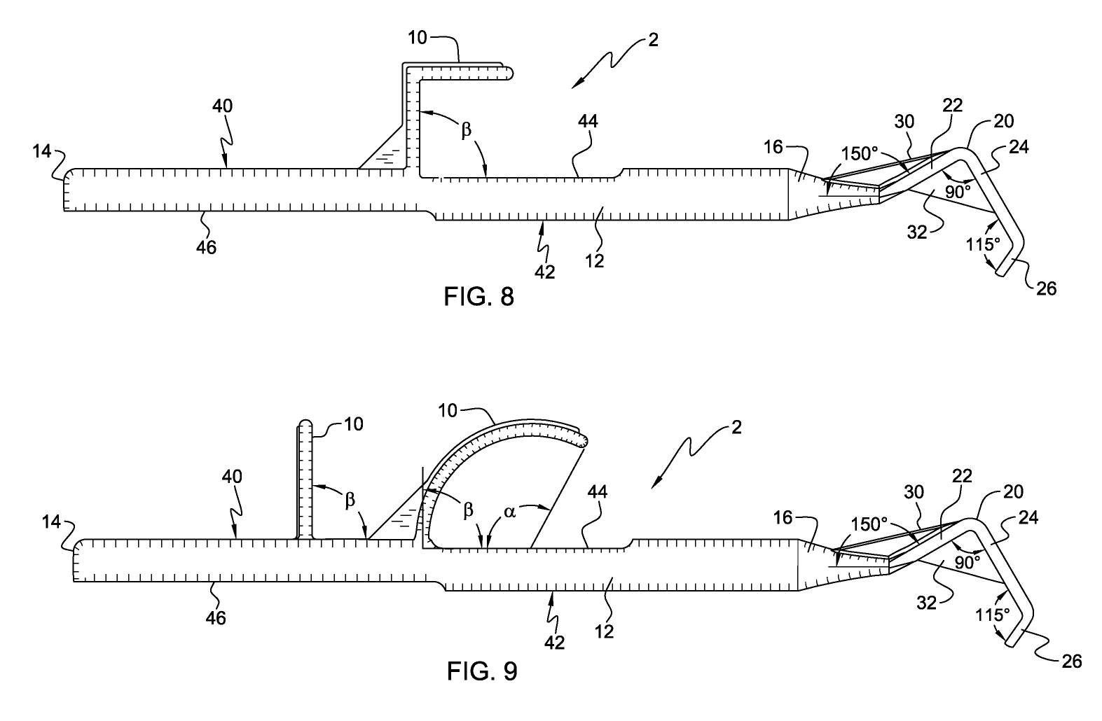

In the embodiment shown in FIGS. 1A and 4A, finger member 10 is arcuate. In alternative aspects of the invention finger member 10 may be a number of different embodiments. Finger member 10 may be a non-arcuate member attached to shaft 12. For example, finger member 10 may be a substantially straight member attached to shaft 12. Referring now to FIG. 6, finger member 10 is shown there as a straight member attached to shaft 12, the angle β formed therebetween is substantially 90°. Now referring to FIG. 7, finger member 10 is shown as a straight member attached to shaft 12 the angle β formed thereby is substantially 30°. The finger member 10 shown in FIG. 7 is one example of an angled finger member. Alternatively, finger member 10 may consist of more than one connected member. For example, as shown in FIG. 8, finger member 10 may consist of two connected members. If finger member 10 consists of multiple members, those members need not approximate the arcuate segment shown in FIGS. 1A and 4A. In FIG. 4A, the angle β is formed between shaft 12 and finger member 10 at the point of contact therebetween. For example purposes, the angle shown in FIG. 4A is about 90°. It is contemplated though that angle β could range from 30° to 150°, inclusive. This range is contemplated for all possible embodiments, including, for example, where finger member 10 is arcuate, where finger member 10 is comprised of a single non-arcuate member, and where finger member 10 is comprised of multiple members. It is also contemplated that a plurality of finger members may be attached to shaft 12. For example, FIG. 9 shows two finger members 10 attached to shaft 12. One finger member shown in FIG. 9 is arcuate, while the other is a straight member. It is contemplated that, each finger member 10 in the plurality could have any of the shapes described above. In FIG. 9, the finger members 10 are shown aligned for example purposes. However, it is contemplated that the finger members 10 may not be aligned with one another.

Referring specifically to FIG. 4A, the angle α is formed between the end or tip of finger member 10 and shaft 12. More specifically, angle α is formed about a location on shaft 12 substantially 0.6 inches closer to proximal end 16 than finger member 10. However, it is contemplated that angle α could be formed about a location along shaft 12 ranging from 0.2 inches to 1 inch closer proximal end 16 than finger member 10. It is contemplated that angle α may be any angle between 0° and 180°, inclusive. For example purposes, the angle α shown in FIG. 4 is about 120°, and formed about a point approximately 0.6 inches closer to proximal end than finger member 10.

Finger member 10, shown in FIGS. 1A and 4A, may be made of several different materials. For example, finger member 10 may be made of hard plastic, metal, or any other inflexible material suitable for sterilization. Additionally, finger member 10 may be a manipulable plastic, capable of retaining a form that it is given.

In FIGS. 1A, 2 and 4A, finger member 10 is shown as integrally attached to shaft 12. It is contemplated that finger member 10 may be removably attached as shown in FIG. 4D. In the aspect of the invention where finger member 10 is removably attached, it is contemplated that finger member 10 may be attached at any location along shaft 12. It is further contemplated that finger member 10 may be attached at distal end 14 or at proximal end 16. Possible means for attaching finger member 10 to shaft 12 are contemplated to include, but are not limited to, a clip, possibly a C-shaped clip, an O-ring, sized appropriately for the cross-section of shaft 12, a threaded screw, or a rail. For example, FIG. 4D shows an exploded view of finger member 10 attached to a C-shaped clip 70, which may be attached to shaft 12. FIG. 4D shows C-shaped clip 70 sized appropriately for the cross section of shaft 12 so that C-shaped clip 70 may be secured for temporary usage, and then removed.

It is also contemplated that finger member 10 may be movably positioned and/or secured as shown in FIG. 4E. In this aspect of the invention, finger member 10 may move along shaft 12 without being detached. It is contemplated that finger member 10 may move either axially along or rotationally around shaft 12. Finger member 10 may be movably attached to shaft 12 by, but not limited to, a rail or an O-ring appropriately sized to the cross-section of shaft 12. It is contemplated that after finger member 10 has been moved to the desired location along shaft 12, finger member 10 may be locked in place. Referring specifically to FIG. 4E, a rail 80 is attached to superior surface 40. Finger member 10 is attached to rail 80 such that finger member 10 may move freely along rail 80. It is contemplated that a threaded screw 82 is attached to finger member 10. Finger member 10 may be temporarily fixed in place along rail 80 and shaft 12 by the tightening of threaded screw 82. FIG. 4E shows rail 80 on superior surface 40. However, it is contemplated that rail 80 could be located on any surface along shaft 12. FIG. 4E also shows rail 80 in a primarily axial direction. It is also contemplated, however, that rail 80 could circumscribe shaft 12.

It is also contemplated that finger member 10 may be movably affixed to shaft 12 without being locked in place. For example, finger member 10 may be attached to a spring within shaft 12. When pressure is placed against finger member 10, the spring may compress, causing the finger member to move along shaft 12. Thus, the displacement of finger member 10 is greater than the overall displacement of surgical retractor 2. The sensitivity of surgical retractor 2 will therefore be enhanced. Further, it is contemplated that springs of different strengths may be included in surgical retractor 2.

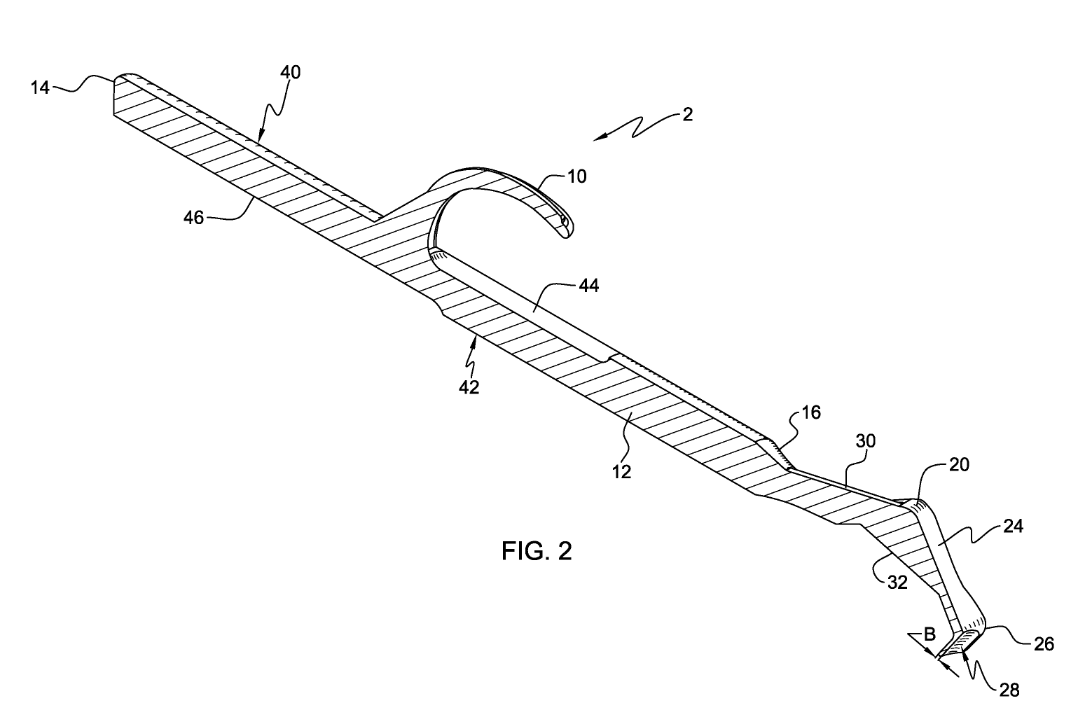

In FIG. 1A, shaft 12 is shown as a cylinder with a circular cross-section. However, shaft 12 may have a different cross-sectional shape, including, for example, hexagonal, trapezoidal, oval, or polygonal to allow for close approximation to tissue, or for comfort in the hand of the user. Shaft 12 has a superior surface 40 and an inferior surface 42. Referring specifically to FIGS. 2 and 4A, Shaft 12 has a grip region 46 on inferior surface 42. In the embodiment shown in FIGS. 2 and 4A, grip region 46 extends from distal end 14 to a location immediately below the projection of finger member 10 on inferior surface 42. However, it should be noted that grip region 46 could be omitted from the invention entirely. Alternatively, grip region 46 could extend from distal end 14 to proximal end 16. In yet another embodiment, grip region 46 could extend from any location on surface 42 to any other location on surface 42.

In the embodiment shown in FIGS. 2, and 4A, grip region 46 is shown as a relatively flattened portion of shaft 12, for ease of holding and manipulation. Alternative grip regions are contemplated to include, for example, a covering, a textured surface, or knurled surface. The covering may be, but is not limited to a chemical spray or coating that may be applied, or a sheath of a separate material. Contemplated texturing includes, but is not limited to a ribbing or cross-hatching. It is also contemplated that grip region 46 may extend beyond inferior surface 42. It is contemplated that grip region 46 may extend to any location between inferior surface 42 and superior surface 40. In an alternative embodiment, grip region may circumscribe shaft 12, covering inferior surface 42, superior surface 40, and the exterior of shaft 12 between inferior surface 42 and superior surface 40. It is also contemplated that grip region 46 may be a modification of shaft 12 in multiple ways to further increase the ease of holding and manipulation. For example grip region may be a textured, flattened region. Another alternative contemplated includes a flattened region that has also been covered with a sheath or chemical spray. These modifications are illustrations; other combinations of modifications to shaft 12 will be recognized by those skilled in the art.

Referring to FIGS. 2 and 4A, superior surface 40 has a grip region 44. In the embodiments shown, grip region 44 extends from finger member 10 along shaft 12 on superior surface 40 to a location substantially midway between finger member 10 and proximal end 16. Alternatively, grip region 44 could extend from finger member 10 to proximal end 16. Those skilled in the art will recognize that grip region 44 may extend from any location closer to proximal end 16 than finger member 10 to any location up to and including proximal end 16. In the embodiment shown in FIGS. 2 and 4A, grip region 44 is a relatively flattened area of shaft 12. Contemplated alternatives to the flattened surface, to provide friction and ease of manipulation to the user include, but are not limited to, a covering, a textured surface, or a knurled surface. A covering, a textured surface and a knurled surface for grip region 44 are defined as they are above for grip region 46. It is further contemplated that grip region 44 may extend beyond superior surface 40 to any location in the direction of inferior surface 42. It is also contemplated that grip region 44 may circumscribe shaft 12. Additionally, grip region 44 may be modified in multiple ways, as described above for grip region 46.

Proximal end 16 may be tapered, as shown in FIGS. 1A and 4A. Alternatively, proximal end 16 may be flat and not tapered. If proximal end 16 is tapered, rake head 20 is attached at the small end of the taper as shown in FIGS. 1A and 4A. In the embodiment of the surgical retractor shown, rake head 20 is comprised of a first segment 22, a second segment 24 and a third segment 26. First segment 22 is attached to proximal end 16, second segment 24 is attached to first segment 22, and third segment 26 is attached to third segment 26.

In the embodiment shown in FIG. 4, for example purposes a 150° angle is shown between first segment 22 and shaft 12, however it is contemplated that this angle may vary from 120° and 180°, inclusive. For example purposes, the angle between first segment 22 and second segment 24 is shown as 90°, but it is contemplated that it may vary from 60° and 180°, inclusive. For example purposes, the angle between second segment 24 and third segment 26 is 115°, but it is contemplated that it may vary from 85° to 145°, inclusive. Rake head 20 is configured to retract the desired tissue. In FIG. 4, the embodiment shows each of these angles as fixed. It is contemplated, however, that each of these angles may be adjustable by the user to customize the rake head 20 pitch and reach. FIG. 4B, for example, shows rake head 20 in detail where rake head 20 is adjustable. The junction of first segment 22 and shaft 12 is shown connected with a screw 50. The junction of first segment 22 and second segment 24 is shown connected with a screw 52. The junction of second segment 24 and third segment 26 is shown connected with a screw 54. Each of the aforementioned angles may be adjusted by turning the screw, thereby adjusting the angle. Screw 50, screw 52, and screw 54 are shown for example purposes, and it is contemplated that screw 50, screw 52, and screw 54 may be replaced by other connection means, including, for example, pins, bolts, adhesives, and clips.

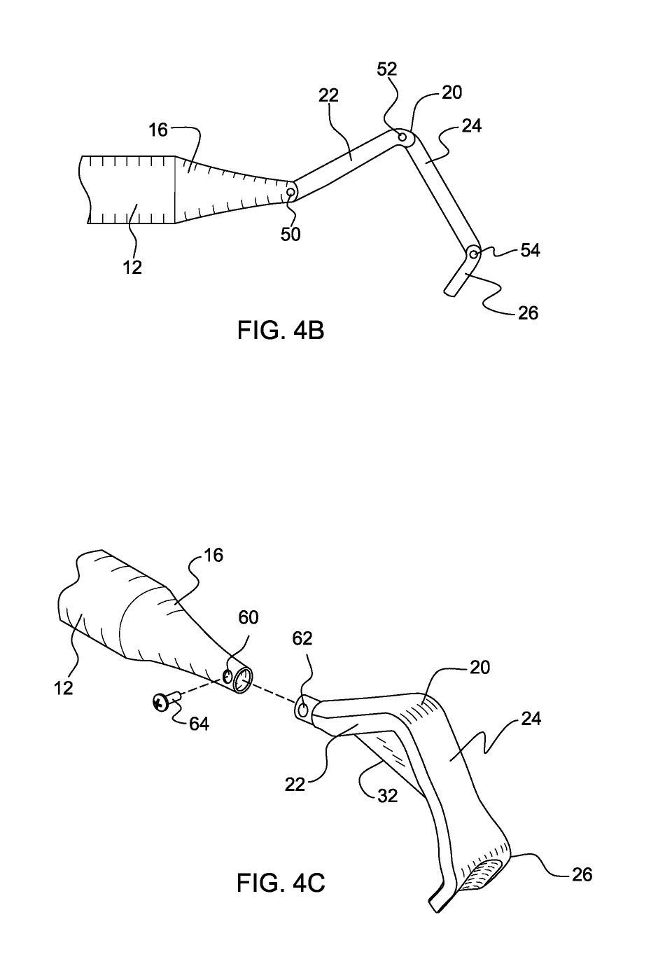

In the embodiments show in FIGS. 1A and 4A, the first segment 22, second segment 24 and third segment 26 have relatively planar configurations relative to the longitudinal axis of shaft 12. First segment 22, second segment 24, and third segment 26 are shown with rectangular cross-sections. It is contemplated that the cross-sections of first segment 22 and third segment 24 could have additional cross-sections, including but not limited to, circular, oval, and polygonal for alternative applications. It is further contemplated that different cross-sectional sizes could be utilized for first segment 22 and second segment 24 depending on the particular clinical application. In the embodiment shown in FIGS. 1A and 5, second segment 24 is shown as being tapered relative to the coronal plane. This taper may be present with any of the cross-sections including for first segment 22 and second segment 24 depending on the clinical application. One skilled in the art would recognize that first segment 22 and second segment 24 may be varied in additional ways. For example, first segment 22 and second segment 24 may be arcuate. Also as an example, rather than being two discrete segments, first segment 22 and second segment 24 may be aligned in the same direction, the angle formed between them being 180°, ultimately forming one member. It is further contemplated that first segment 22 may be oriented along the same axis as shaft 12, the angle formed between them being 180° such that they form one segment. One skilled in the art would also recognize that first segment 22 may be replaced by multiple segments or that second segment 24 may be replaced by multiple segments.

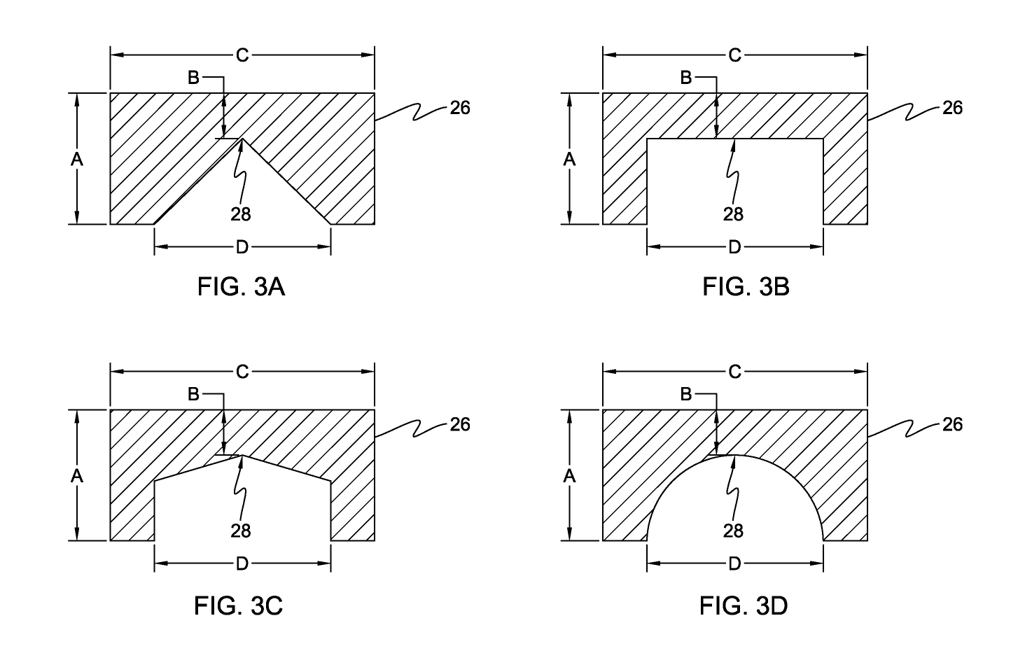

As seen in FIGS. 1A, 1B, 2 and 5, third segment 26 has an indentation 28 or relief formed upon it. In FIGS. 1A, 1B, 2 and 5, indentation 28 is a concavity that allows the user to avoid collateral tissue impingement when surgical retractor 2 is in use. An example of one cross-section of the concavity is shown in FIG. 3D. It is contemplated that indentation 28 could take on other shapes, including, for example purposes, indentation 28 could be rectangular, arcuate, angled, triangular or polygonal depending on the clinical application and other instruments used in the procedure. For example: FIG. 3A shows a triangular cross-section for indentation 28; FIG. 3B shows a rectangular cross-section for indentation 28; FIG. 3C shows a polygonal cross-section for indentation 28. The indentation cross-sections in FIGS. 3A-D are shown for example purposes only; one skilled in the art would recognize that additional cross-sections for indentation 28 are possible. It is also contemplated that in replacement of indentation 28, a surface treatment or roughness from manufacturing could be used to facilitate tissue capture.

FIG. 5 shows third segment 26 as having a generally rectangular shape to facilitate inserting into the surgical opening. Alternative configured embodiments are contemplated; for example, third segment 26 could be triangular, semi-circular, circular, oval, trapezoidal, hexagonal or polygonal, that may be preferable depending on the operation.

Referring to FIGS. 1B and 3A-D, third segment 26 has a maximum depth A, which is shown as 0.04 inches for example purposes, but it is contemplated that this depth may vary from 0.1 inches to 0.01 inches. Referring to FIGS. 2 and 3D, third segment 26 has a minimum depth B, corresponding to the deepest location of indentation 28. B is approximately 0.015 inches as shown in FIGS. 2 and 3D, but it is contemplated that depth B could range from 0.085 inches to 0.005 inches. The relative sizes of A and B are listed for example purposes only, additional sizes may occur to one skilled in the art depending on the procedure. Depth A and depth B may be varied based on the surgical procedure involved, the size of the incision, the nature of the tissue retracted, or the other instruments to be used during the procedure.

Referring now to FIGS. 3A-D and 5, third segment 26 has a width C, and indentation 28 has a width D. Width C is approximately 0.236 inches and width D is approximately 0.141 inches, as shown in FIG. 5. However, one skilled in the art will recognize that that width C and width D may be varied. Width C may range from 1 inch to 0.1 inches, and width D may range from 0.9 inches to 0.05 inches based upon the procedure involved, the size of the incision, the nature of the tissue retracted, or the other instruments to be used during the procedure.

As seen in FIGS. 1A and 4A, rake head 20 may be strengthened with a first support 30 that is attached to proximal end 16 and first segment 22. A second support 32 may be added to support rake head 20. Second support 32 attaches to first segment 22 and second segment 24. Although not shown, surgical retractor 2 may be configured without both first support 20 and second support 32, depending on the end use of surgical retractor 2. In another alternative embodiment, surgical retractor 2 may have a first support 30, but not a second support 32 or vice versa.

The embodiment shown in FIGS. 1A and 4A, rake head 20 is integrally attached to shaft 12. However, as shown in FIG. 4C, in an alternative embodiment rake head 20 may be detachable from shaft 12. The detachment of rake head 20 may occur immediately at the junction of proximal end 16 and rake head 20 as shown in FIG. 4C. Alternatively, proximal end 16 may be detachable along with rake head 20. Potential means for attaching rake head 20 to shaft 12 include, for example purposes, a threaded screw, a snap-lock, a locking hinge, slide rails, a press-fit lock and a spring lock. For example, FIG. 4C shows proximal end 16 and rake head 20 in an embodiment where rake head 20 is detachable. In the embodiment shown in FIG. 4C, first segment 22 can be inserted into proximal end 16. A first aperture 60 is formed on proximal end 16. A second aperture 62 is formed on first segment 22. A threaded screw 64 may be inserted through first aperture 60 and second aperture 62, thereby securing first segment 22 to proximal end 16. Contemplated alternatives to threaded screw 64 include, but are not limited to a bolt and a pin.

Generally, the method of using a surgical retractor is disclosed herein, and many alterations would occur to one skilled in the art, and are contemplated as a part of the invention. This description of the procedure should be understood to encompass all possible embodiments of the surgical retractor previously discussed. The method for using the surgical retractor includes, generally, inserting a rake head of a surgical retractor into an opening, such as the surgical slicing of a human or an animal, or alternatively within a wound site and adjacent tissue, placing a digit against a finger member, urging the surgical retractor in a primarily axial direction, and engaging the edge of the opening. A digit may be a finger or a thumb. Those skilled in the art would recognize that multiple digits may be placed against the finger member rather than a single digit.

One skilled in the art will recognize that the following references to medial and lateral are made for illustrative purposes only, and that surgical retractor 2 may be used to retract tissue from either a medial side or a lateral side. Similarly, one skilled in the art will recognize that an opening may be located anywhere on the body, and the directional orientation discussed below are for example purposes only.

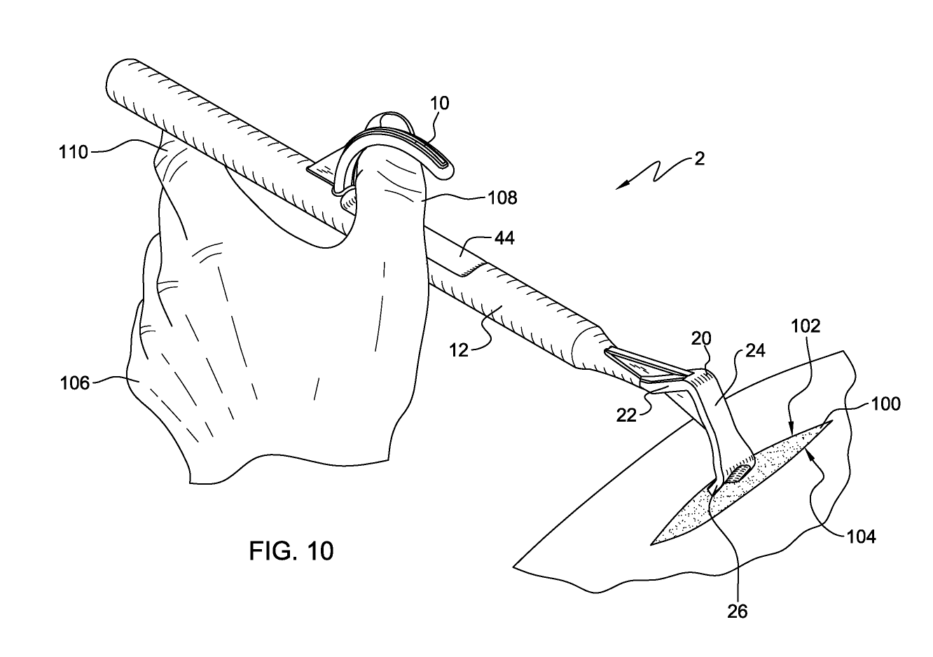

Referring to FIG. 10, rake head 20 of surgical retractor 2 is shown inserted into an opening 100. Opening 100 has a medial side 102 and a lateral side 104. Rake head 20 is shown contacting medial side 102, such that shaft 12 extends in a primarily medial direction.

Still referring to FIG. 10, a hand 106 is shown engaging surgical retractor 2. However, it should be noted that hand 106 may grasp surgical retractor 2 or may merely rest adjacent surgical retractor 2. A digit 108 is shown against finger member 10. Digit 108 is shown as the thumb in FIG. 10. It is contemplated that any digit, including the pinky, ring, middle, index or thumb, may be placed against finger member 10. It is also contemplated that finger member 10 may be configured to accommodate more than one digit may be placed against finger member 10 or that the side of the hand or wrist may be placed against finger member 10. Still referring to FIG. 10, a digit 110 is shown supporting shaft 12, such that inferior surface 42 rests upon digit 110. In FIG. 10, digit 110 is shown as the index finger. It is contemplated that in alternative methods, digit 110 may be the pinky, ring, middle, index or thumb. It is also contemplated that multiple digits may support shaft 12. It is also contemplated that one or more digits may rest upon superior surface 40, while digit 110 supports shaft 12.

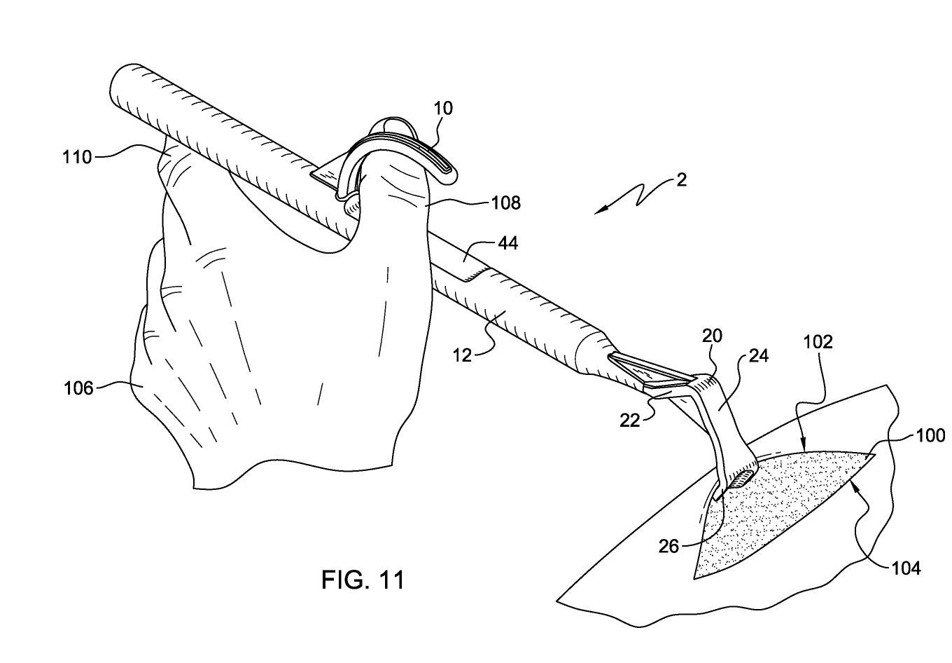

Referring now to FIG. 11, rake head 20 of surgical retractor 2 is shown inserted into opening 100 with digit 108 resting against finger member 10. Surgical retractor 2 has been urged in an axial, medial direction. Thus medial side 102 and therefore opening 100 have been engaged and expanded. The distance between medial side 102 and lateral side 104 has increased.

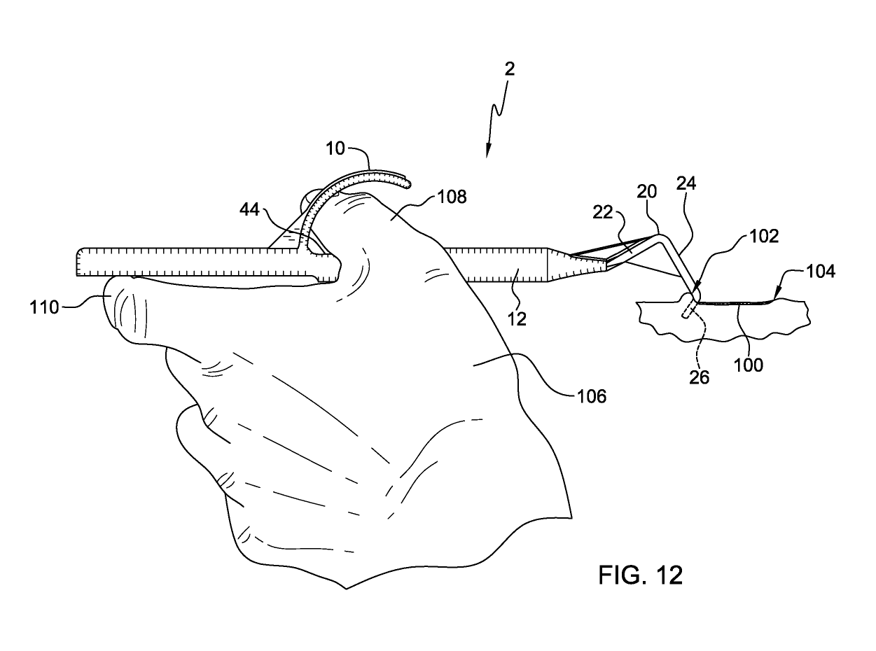

Now referring to FIG. 12, rake head 20 is shown engaged to medial side 102. Third segment 26 rests proximate medial side 102 and second segment 24 rests above medial side 104. The junction of third segment 26 and second segment 24 engages medial side 102.

The above steps are intended to be illustrative and do not contain all of the possible steps that may be incorporated into the method as one skilled in the art would recognize that the order of many of these steps is also interchangeable.

The method of use of surgical retractor 2 may also include selecting finger member 10 from an assortment of attachable finger members. The following step may be attaching selected finger member 10 to shaft 12. The step of attaching finger member 10 to shaft 12 may include a threaded screw, a snap-lock, a locking hinge, a slide rail, a press-fit lock, or a spring lock.

Further, an additional step (not shown) may include moving finger member 10 along shaft 12 to a desired location. Then, it is contemplated that finger member 10 will be locked into place along shaft 12. Movement of finger member 10 may be accomplished along a rail attached to shaft 12, or an O-ring attached to finger member 10 surrounds shaft 12, and allows finger member 10 to slide freely until locked.

Another step (not shown) may include selecting an appropriate rake head 20. The selected rake head 20 may then be attached to proximal end 16. The step of attaching rake head 20 may include, for example, a threaded screw, a snap-lock, a locking hinge, a slide rail, a press-fit lock, or a spring lock.

A further step (not shown) may include adjusting the angle between first segment 22 and shaft 12. Yet another step may include adjusting the angle between first segment 22 and second segment 24. Still another step may include adjusting the angle between second segment 24 and third segment 26. These adjustment steps allow the user to configure rake head 20 to have the appropriate reach and pitch.

It is also contemplated that as surgical retractor 2 is urged in the primarily axial and medial direction that finger support 10 may move along shaft 12. This step of the method is accomplished by the compression or expansion of a spring or other dampening device, which alters the movement of finger member 10 in relation to shaft 12.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A surgical retractor for expanding an incision in a living being, comprising:
a shaft, with a proximal end and a distal end, a superior surface and an inferior surface;
a finger member, attached to the shaft intermediate of the distal end and the proximal end;
a rake head, connected to the proximal end and configured to facilitate entering into an incision;
wherein the finger member is further configured to accommodate a guiding digit and the shaft is configured to be gripped by a single hand and be pulled in a primarily axial direction; and
wherein the surgical retractor is configured to expand an incision in a living being.

2. The surgical retractor of claim 1, wherein the finger member is fixed to the shaft.

3. The surgical retractor of claim 1, wherein the finger member is movably attached along the shaft.

4. The surgical retractor of claim 1, wherein the finger member is removably attached to the shaft.

5. The surgical retractor of claim 1, wherein the finger member is arcuate.

6. The surgical retractor of claim 1, wherein the finger member is non-arcuate.

7. The surgical retractor of claim 6, wherein the finger member is angled.

8. The surgical retractor of claim 1, wherein the finger member is comprised of multiple members.

9. The surgical retractor of claim 1, further comprising:
a plurality of finger members.

10. The surgical retractor of claim 1, wherein the rake head is integrally attached to the proximal end of the shaft.

11. The surgical retractor of claim 1, wherein the rake head is removably attached to the proximal end of the shaft.

12. The surgical retractor of claim 1, wherein the rake head is further comprised of:
a first segment attached to the shaft, a first angle formed thereby;
a second segment attached to the first segment a second angle formed thereby;
a third segment attached to the second segment a third angle formed thereby; and wherein the third segment has a depression formed thereon.

13. The surgical retractor of claim 12, wherein the depression is a concavity.

14. The surgical retractor of claim 12, wherein:
the first angle is fixed;
the second angle is fixed; and
the third angle is fixed.

15. The surgical retractor of claim 12, wherein:
the first angle is adjustable;
the second angle is adjustable; and
the third angle is adjustable.

16. The surgical retractor of claim 12, wherein:
the first angle is substantially 150°;
the second angle is substantially 90°; and
the third angle is substantially 115°.

17. A method of using the surgical retractor, comprising:
obtaining a surgical retractor, the surgical retractor comprising:
a shaft, with a proximal end and a distal end, a superior surface and an inferior surface;
a finger member, configured to accommodate a guiding digit, attached to the shaft intermediate of the distal end and the proximal end; and
a rake head, connected to the proximal end and configured to facilitate entering into an incision.
inserting the rake head into an opening;
placing a digit against the finger member;
urging the surgical retractor in a primarily axial direction; and
engaging the opening, to expand the opening.

18. The method of claim 17, further comprising:
selecting the finger member from a plurality of finger members;
attaching the finger member to the shaft;
selecting the rake head from a plurality of rake heads; and
attaching the rake head to the proximal end of the shaft

19. A surgical retractor used for opening an incision in a living being, comprising:
a cylindrical shaft having a distal end, a proximal end, a superior and inferior surface, wherein the proximal end is tapered;
a finger member, configured to accommodate a guiding digit, attached to the shaft substantially midway between the distal end and the proximal end on the superior surface, wherein the finger member is arcuate; and
a rake head, configured to facilitate entering into an incision, attached to the proximal end, wherein the rake head is comprised of a first segment, attached to the shaft, forming an angle of substantially 150°, a second segment attached to the first segment, forming an angle of substantially 90°, and a third segment attached to the second segment, forming an angle of substantially 115°.
